(19) United States
(12) Patent Application Publication
(10) Pub. No.: US 2025/0115541 A1
(43) Pub. Date: Apr. 10, 2025

(54) METHOD FOR PREPARING VERATROLE

(71) Applicant: SHANDONG HOLLY PHARMACEUTICAL CO., LTD., Binzhou (CN)

(72) Inventors: Huaqiang Wu, Binzhou (CN); Lanhua Li, Binzhou (CN); Yuenan Qiu, Binzhou (CN); Zhiyuan Liu, Binzhou (CN); Hongyuan Zhang, Binzhou (CN); Lijun Jiang, Binzhou (CN); Zhen Lin, Binzhou (CN); Shuqiang Gai, Binzhou (CN); Kuankuan Sun, Binzhou (CN)

(73) Assignee: SHANDONG HOLLY PHARMACEUTICAL CO., LTD., Binzhou (CN)

(21) Appl. No.: 18/257,285

(22) PCT Filed: Dec. 20, 2022
(86) PCT No.: PCT/CN2022/140231

§ 371 Date: Aug. 23, 2024

(30) Foreign Application Priority Data
Jan. 17, 2022 (CN) 202210046445.8

(51) Int. Cl. C07C 43/205

(52) U.S. Cl. CPC C07C 43/2055

(57) ABSTRACT

The disclosure relates to the technical filed of organic synthesis, in particular to a method for preparing veratrole. In the disclosure, veratrole is prepared by using lithium hydroxide instead of sodium hydroxide, catechol as raw material, and dimethyl sulfate as methylating agent. Comparing with the existing method of adding sodium hydroxide solution, the method according to the disclosure allows reduced reaction temperature, shortened reaction time, and thereby improves the stability and safety of the reaction and the product yield. The experimental data of the examples in the disclosure shows a veratrole yield up to 95%. Furthermore, the preparation method according to the disclosure does not need to be carried out under a reflux condition, the operation of which is simpler, thereby further improving the safety and stability of the reaction.

### CROSS REFERENCE TO RELATED APPLICATION

This application claims the benefit and priority of Chinese Patent Application CN202210046445.8, entitled “Method for preparing veratrole” filed on Jan. 17, 2022, the disclosure of which is incorporated by reference herein in its entirety as part of the present application.

### TECHNICAL FIELD

The present disclosure relates to the technical filed of organic synthesis, in particular to a method for preparing veratrole.

### BACKGROUND ART

Heart disease is a disease that seriously endangers human health. According to statistics, there are about 370 million patients suffering from various heart diseases in China, and hundreds of thousands of people die of heart disease every year. Also, there are also about 3 million new patients of heart disease every year. In China, the onset age of heart disease patients also shows a trend to the younger, which has led to an increase in the variety of drugs for treating various types of heart disease year by year.

Veratrole, also with a chemical name of 1,2-dimethoxy benzene, has a melting point of 22-23° C., pure product of which is a colorless transparent liquid and becomes solid at low temperature. It is an important fine chemical raw material and one of the important raw materials in the pharmaceutical field. It is not only a key intermediate in the synthesis of bactericide of dimethomorph, but also an important intermediate in the synthesis of drugs for treating heart disease.

The existing methods for synthesizing veratrole mainly include: conducting a methylation in NaOH solution to prepare crude product by using catechol as raw material and dimethyl sulfate as methylating agent; then subjecting the crude product to a water washing and an alkali washing; and finally separating out a final product by distillation. This synthesis method requires a reaction temperature of at least 99° C. and a long reaction time of least 8 hours under reflux, which is not conducive to the stability and safety of the synthesis reaction.

### SUMMARY

In view of this, the present disclosure provides a method for preparing veratrole, which allows a low reaction temperature and short reaction time, improves the stability and safety of the reaction, and has a high product yield.

In order to achieve the above-mentioned object, the present disclosure provides the following technical solutions.

Provided is a method for preparing veratrole, including the steps of:
mixing catechol, dimethyl sulfate and lithium hydroxide to obtain a mixture; and
subjecting the mixture to a methylation reaction to obtain veratrole,
wherein the methylation reaction is conducted at a temperature of not higher than 80° C. for a time of not longer than 6 h.

In some embodiments, the methylation reaction is conducted at the temperature of 60-80° C. for the time of 2-6 h.

In further embodiments, a mass ratio of catechol, dimethyl sulfate and lithium hydroxide is in a range of 1:(1-1.3):(0.92-1.3).

In some embodiments, mixing catechol, dimethyl sulfate and lithium hydroxide comprises:
mixing catechol and dimethyl sulfate to obtain a reaction solution; and
adding a lithium hydroxide solution dropwise into the reaction solution to obtain the mixture.

In some embodiments, a mass percentage of lithium hydroxide in the lithium hydroxide solution is in a range of 30% to 50%.

In some embodiments, the lithium hydroxide solution is prepared from lithium hydroxide monohydrate, and a solvent of the lithium hydroxide solution is water.

In some embodiments, the lithium hydroxide solution is added dropwise in two batches.

In some embodiments, adding the lithium hydroxide solution dropwise is performed by the following step of:
heating the reaction solution;
starting to add dropwise the lithium hydroxide solution into the reaction solution at a time when a temperature of the reaction solution reaches 10° C.-40° C., such that not less than 50% of a total amount of the lithium hydroxide solution to be added dropwise has been added by a time when the temperature of the reaction solution reaches a temperature required for the methylation reaction; and
continuing to add dropwise a remaining lithium hydroxide solution thereto within 1.5-4.0 h.

In some embodiments, the method further comprises, after the methylation reaction, conducting a water washing, an alkali washing and a distillation in sequence on a product mixture obtained from the methylation reaction.

In some further embodiments, the water washing is performed by
leaving the product mixture obtained from the methylation reaction to stand and stratify, to obtain an oil layer; and
conducting the water washing on the oil layer.

In some embodiments, a reagent for the alkali washing is one selected from the group consisting of a LiOH solution, a NaOH solution, a KOH solution, and a NaCO₃ solution.

In some embodiments, the method further comprises, after the alkaline washing, neutralizing a product obtained from the alkaline washing to a pH value of 1 with hydrochloric acid.

In some embodiments, the distillation is conducted at a vacuum degree of −0.09998 Mpa to −0.0998 Mpa.

The present disclosure provides a method for preparing veratrole, including the steps of: mixing catechol, dimethyl sulfate and lithium hydroxide to obtain a mixture; and subjecting the mixture to a methylation reaction to obtain veratrole; wherein the methylation reaction is conducted at a temperature of not higher than 80° C. for a time of not longer than 6 h. In the present disclosure, veratrole is prepared by using lithium hydroxide instead of sodium hydroxide, catechol as raw material, and dimethyl sulfate as methylating agent. Comparing with the existing method of adding sodium hydroxide solution, the method according to the present disclosure allows reduced reaction temperature, shortened reaction time, and thereby improves the stability and safety of the reaction and the product yield. The experimental data of the examples in the present disclosure shows a yield of up to 95%.

Furthermore, the preparation method according to the present disclosure does not need to be carried out under a reflux condition, the operation of which is simpler, thereby further improving the safety and stability of the reaction.

### DETAILED DESCRIPTION OF THE EMBODIMENTS

The present disclosure provides a method for preparing veratrole, including the steps of:
mixing catechol, dimethyl sulfate and lithium hydroxide to obtain a mixture; and
subjecting the mixture to a methylation reaction to obtain veratrole; wherein the methylation reaction is conducted at a temperature of not higher than 80° C. for a time of not longer than 6 h.

Unless otherwise specified, the raw materials used in the present disclosure are all commercially available.

In some embodiments, the methylation reaction is carried out in a methylation reactor at a temperature of not higher than 80° C., preferably 60° C.-80° C., more preferably 70° C.-75° C., and most preferably 75° C. In some embodiments, the time of the methylation reaction is timed from the completion of the mixing catechol, dimethyl sulfate and lithium hydroxide. The methylation reaction is carried out for less than or equal to 6 h, preferably 2-6 h, more preferably 3-5 h. further more preferably 4-5 h, and most preferably 5 h. In some embodiments, catechol, dimethyl sulfate and lithium hydroxide are mixed by stirring and then subjected to the methylation reaction.

In the present disclosure, catechol, dimethyl sulfate and lithium hydroxide are mixed to undergo the methylation reaction. In some embodiments, a mass ratio of catechol, dimethyl sulfate and lithium hydroxide is in a range of 1:(1-1.3):(0.92-1.3), preferably 1:(1-1.20):(0.95-1.25), more preferably 1:(1-1.15):(0.95-1.2), and most preferably 1:1.14:1.2. In the present disclosure, the mass ratio of catechol, dimethyl sulfate and lithium hydroxide is limited within the above ranges, which is beneficial to improving catalytic effect of lithium hydroxide and thereby increasing the yield of veratrole.

In some embodiments, a step of the mixing catechol, dimethyl sulfate and lithium hydroxide comprises: mixing catechol and dimethyl sulfate to obtain a reaction solution, and then adding a lithium hydroxide solution dropwise into the reaction solution to obtain the mixture. In some embodiments, a mass percentage of lithium hydroxide in the lithium hydroxide solution is in a range of 30% to 50%, preferably 40% to 50%, more preferably 40% to 45%, and most preferably 40%; and a solvent of the lithium hydroxide solution is water. In some embodiments, the lithium hydroxide solution is prepared from lithium hydroxide monohydrate. In the present disclosure, due to lower price than anhydrous lithium hydroxide, the reaction cost is reduced by using lithium hydroxide monohydrate. In the present disclosure, the process for preparing the lithium hydroxide monohydrate solution is not particularly limited, and it is preferable to dissolve the lithium hydroxide monohydrate in deionized water to prepare the lithium hydroxide solution.

In some embodiments, the lithium hydroxide solution is added dropwise in two batches. In some embodiments, the lithium hydroxide solution is added dropwise by the following steps: heating the reaction solution; starting to add dropwise the lithium hydroxide solution into the reaction solution at a time when the temperature of the reaction solution reaches 10° C.-40° C., such that not less than 50% of a total amount of the lithium hydroxide solution to be added dropwise has been added by a time when the temperature of the reaction solution reaches a temperature required for the methylation reaction; and then continuing to add dropwise a remaining lithium hydroxide solution thereto within 1.5-4.0 h. In some embodiments, adding dropwise the lithium hydroxide solution into the reaction solution is started at a time when the temperature of the reaction solution reaches 10° C.-40° C., preferably 15° C.-35° C., more preferably 20° C.-30° C., and most preferably 25° C. In the present disclosure, preferably the lithium hydroxide solution is started to be added dropwise into the reaction solution at the above-defined temperature, so that the temperature does not rise too quickly and is easy to control, resulting in that the reaction temperature is relatively stable so as to smooth the reaction procedures, thereby guaranteeing the stability of final product yield. By a time when the temperature of the reaction solution reaches a temperature required for the methylation reaction, the lithium hydroxide solution has been added dropwise in an amount of not less than 50%, preferably 50%-60%, and more preferably 60% of a total amount of the lithium hydroxide solution to be added dropwise. In the present disclosure, since the methylation reaction is an exothermic reaction, the heat released by the exothermic reaction can be used to raise the temperature of the reaction solution so that the temperature of the reaction solution reaches the temperature required for the methylation reaction. In some embodiments, circulating water is introduced to prevent the temperature of the reaction solution from exceeding the temperature required for the methylation reaction. In some embodiments of the present disclosure, when continuing adding the remaining lithium hydroxide solution dropwise, the adding dropwise of the remaining lithium hydroxide solution is completed within 1.5 h to 4.0 h, preferably 1.5 h to 3.0 h, more preferably 1.5 h to 2.5 h, and most preferably 2.0 h. Because the methylation reaction of the remaining lithium hydroxide solution releases less heat, preferably the time for completing the adding of the remaining lithium hydroxide solution is limited within the above range, which is conductive to avoiding artificial waste during the adding dropwise.

In some embodiments, the method further comprises, after the methylation reaction, conducting a water washing, an alkali washing and a distillation in sequence on a product mixture obtained from the methylation reaction. In some embodiments, after the methylation reaction quits, the product obtained from the methylation reaction is left to stand and stratify to obtain an oil layer, and water washing is conducted on the oil layer. In some embodiments, a process water is used for the water washing. In some embodiments, during the water washing, an organic phase is sampled and detected until the water washing is up to standard. Preferably, an alkali washing is conducted on the product obtained from the water washing. In some embodiments, a reagent for the alkali washing is selected from the group consisting of a LiOH solution, a NaOH solution, a KOH solution and a NaCO₃ solution. In some embodiments, the alkali washing is performed for 1 to 4 times, more preferably for 2 to 4 times, and further more preferably 3 to 4 times. In some embodiments, after the alkaline washing, the method further comprises neutralizing a product obtained from the alkaline washing to a pH value of 1 with hydrochloric acid, and distilling a product after neutralizing. The product obtained from the alkaline washing is neutralized to the above pH value, which can ensure that a metal salt intermediate product formed from the alkali washing is completely converted into the final product. In some embodiments, the distillation is vacuum distillation. In some embodiments, the distillation is conducted at a vacuum degree of −0.09998 Mpa to −0.0998 Mpa, preferably −0.09997 Mpa to −0.0999 Mpa, more preferably −0.09997 Mpa to −0.09993 Mpa, and most preferably −0.09997 Mpa to −0.09994 Mpa.

The technical solution in the present disclosure will be clearly and completely described below in conjunction with the examples in the present disclosure.

### Example 1

1800 g of dimethyl sulfate and 1750 g of catechol were added into a methylation reaction kettle, and then mixed by stirring. When a reaction solution obtained by mixing dimethyl sulfate and catechol was heated up to 30° C., a LiOH solution with a mass percentage of 40% was added dropwise thereto until the added amount of the LiOH solution reached 2100 g. When the temperature of the reaction solution rised up to 70° C., circulating water was turned on, and the remaining LiOH solution was further added dropwise thereto within 1.5 hours. At the end of the adding dropwise, 4200 g of the LiOH solution had been added in total. The resulting reaction solution was subjected to the methylation reaction at 65° C. for 4 hours. After the methylation reaction was completed, the stirring was stopped, and the resulting solution was left standing to make it stratify, obtaining an oil layer. The oil layer was washed with process water until the standard was reached (the organic phase is sampled and detected until the sample was qualified), obtaining a qualified material. After that, the qualified material was subjected to an alkali washing with NaCO₃ three times, and a product obtained from the above alkaline washing was neutralized to a pH value of 1 with hydrochloric acid. The resulting mixture after neutralizing was left to stand and stratify, obtaining a crude oil. The crude oil was distilled under a vacuum condition (a vacuum degree of −0.099935 MPa), obtaining 1605 g of veratrole, with a yield of 92% and a veratrole selectivity of 90%.

### Example 2

1800 g of dimethyl sulfate and 1750 g of catechol were added into a methylation reaction kettle, and then mixed by stirring. When a reaction solution obtained by mixing dimethyl sulfate and catechol was heated up to 25° C., a LiOH solution with a mass percentage of 40% was added dropwise thereto until the added amount of the LiOH solution reached 2310 g. When the temperature of the reaction solution rised up to 70° C. circulating water was turned on, and the remaining LiOH solution was further added dropwise thereto within 3 h. At the end of the adding dropwise, 4200 g of the LiOH solution had been added in total. The resulting reaction solution was subjected to the methylation reaction at 70° C. for 5 h. After the methylation reaction was completed, the stirring was stopped, and the resulting solution was left standing to make it stratify, obtaining an oil layer. The oil layer was washed with process water until the standard was reached (the organic phase is sampled and detected until the sample was qualified), obtaining a qualified material. After that, the qualified material was subjected to an alkali washing with NaCO₃ three times, and a product obtained from the above alkaline washing was neutralized to a pH value of 1 with hydrochloric acid. The resulting mixture after neutralizing was left to stand and stratify, obtaining a crude oil. The crude oil was distilled under a vacuum condition (a vacuum degree of −0.099934 MPa), obtaining 1625 g of veratrole with a yield of 93% and a veratrole selectivity of 89%.

### Example 3

2000 g of dimethyl sulfate and 1750 g of catechol were added into a methylation reaction kettle, and then mixed by stirring. When a reaction solution obtained by mixing dimethyl sulfate and catechol was heated up to 25° C., a LiOH solution with a mass percentage of 50% was added dropwise thereto until the added amount of the LiOH solution reached 2520 g. When the temperature of the reaction solution rised up to 70° C. circulating water was turned on, and the remaining LiOH solution was further added dropwise within 3 h. At the end of the adding dropwise, 4200 g of the LIOH solution had been added in total. The resulting reaction solution was subjected to the methylation reaction at 75° C. for 3 h. After the methylation reaction was completed, the stirring was stopped, and the resulting solution was left standing to make it stratify, obtaining an oil layer. The oil layer was washed with process water until the standard was reached (the organic phase is sampled and detected until the sample was qualified), obtaining a qualified material. After that, the qualified material was subjected to an alkali washing with NaCO₃ three times, and a product obtained from the above alkaline washing was neutralized to a pH value of 1 with hydrochloric acid. The resulting mixture after neutralizing was left to stand and stratify, obtaining a crude oil. The crude oil was distilled under a vacuum condition (a vacuum degree of −0.099937 MPa), obtaining 1660 g of veratrole, with a yield of 95% and a veratrole selectivity of 91%.

### Comparative Example 1

g of dimethyl sulfate and 1750 g of catechol were added into a methylation reaction kettle, and then were mixed by stirring. When a reaction solution obtained by mixing dimethyl sulfate and catechol was heated up to 25° C., a NaOH solution with a mass percentage of 30% was added dropwise thereto until the added amount of the NaOH solution reached 1500 g. When the temperature of the reaction solution rised up to 70° C., circulating water was turned on, and the remaining NaOH solution was further added dropwise thereto within 3 h. At the end of the adding dropwise, 5000 g of the NaOH solution had been added in total. The resulting reaction solution was reacted under reflux at 99° C. for 12 h. After the reaction was completed, the stirring was stopped, and the resulting solution was left standing to make it stratify, obtaining an oil layer. The oil layer was washed with process water until the standard was reached (the organic phase is sampled and detected until the sample was qualified), obtaining a qualified material. After that, the qualified material was subjected to an alkali washing with NaCO₃ three times, and a product obtained from the above alkaline washing was neutralized to a pH value of 1 with hydrochloric acid. The resulting mixture after neutralizing was left to stand and stratify, obtaining a crude oil. The crude oil was distilled under a vacuum condition (a vacuum degree of −0.099939 MPa), obtaining 1500 g of veratrole, with a yield of 86% and a veratrole selectivity of 87%.

The foregoing are only embodiments of the present disclosure, and it should be noted that, to persons of ordinary skill in the art, several modifications and embellishments may be made without departing from the principles of the present disclosure, which are also considered as falling within the protection scope of the disclosure.

What is claimed is:

1. A method for preparing veratrole, comprising the steps of:
mixing catechol, dimethyl sulfate, and lithium hydroxide to obtain a mixture; and
subjecting the mixture to a methylation reaction to obtain veratrole,
wherein the methylation reaction is conducted at a temperature of not higher than 80° C. for a time of not longer than 6 h.

2. The method according to claim 1, wherein the methylation reaction is conducted at the temperature of 60-80° C. for the time of 2-6 h.

3. The method according to claim 1, wherein a mass ratio of catechol, dimethyl sulfate and lithium hydroxide is in a range of 1:(1-1.3):(0.92-1.3).

4. The method according to claim 1, wherein mixing catechol, dimethyl sulfate and lithium hydroxide comprises:
mixing catechol and dimethyl sulfate to obtain a reaction solution; and
adding a lithium hydroxide solution dropwise into the reaction solution to obtain the mixture.

5. The method according to claim 4, wherein a mass percentage of lithium hydroxide in the lithium hydroxide solution is in a range of 30% to 50%.

6. The method according to claim 4 or 5, wherein the lithium hydroxide solution is prepared from lithium hydroxide monohydrate, and a solvent of the lithium hydroxide solution is water.

7. The method according to claim 4 or 5, wherein the lithium hydroxide solution is added dropwise in two batches.

8. The method according to claim 7, wherein adding the lithium hydroxide solution dropwise is performed by
heating the reaction solution;
starting to add dropwise the lithium hydroxide solution into the reaction solution at a time when a temperature of the reaction solution reaches 10° C. to 40° C., such that not less than 50% of a total amount of the lithium hydroxide solution to be added dropwise has been added by a time when the temperature of the reaction solution reaches a temperature required for the methylation reaction; and
continuing to add dropwise a remaining lithium hydroxide solution thereto within 1.5-4.0 h.

9. The method according to claim 1, further comprising, after the methylation reaction, conducting a water washing, an alkali washing and a distillation in sequence on a product mixture obtained from the methylation reaction.

10. The method according to claim 9, wherein the water washing is performed by
leaving the product mixture obtained from the methylation reaction to stand and stratify, to obtain an oil layer; and
conducting the water washing on the oil layer.

11. The method according to claim 9, wherein a reagent for the alkali washing is one selected from the group consisting of a LiOH solution, a NaOH solution, a KOH solution, and a NaCO₃ solution.

12. The method according to claim 9 or 11, further comprising, after the alkaline washing, neutralizing a product obtained from the alkaline washing to a pH value of 1 by using hydrochloric acid.

13. The method according to claim 9, wherein the distillation is conducted at a vacuum degree of −0.09998 Mpa to −0.0998 Mpa.
